(19) United States
(12) Patent Application Publication
(10) Pub. No.: US 2009/0187254 A1
(43) Pub. Date: Jul. 23, 2009

(54) UROLOGICAL MEDICAL DEVICES FOR RELEASE OF UROLOGICALLY BENEFICIAL AGENTS

(75) Inventors: Travis Deal, Freedom, IN (US); James A. Teague, Spencer, IN (US); Shawn C. Bucy, Spencer, IN (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/276,778

(22) Filed: Nov. 24, 2008

Related U.S. Application Data
(60) Provisional application No. 61/008,252, filed on Dec. 19, 2007.

(51) Int. Cl. A61F 2/04; A61M 1/00

(52) U.S. Cl. 623/23.7; 604/8

(57) ABSTRACT

In one aspect, the present invention provides implantable or insertable urological medical devices, which are adapted to release one or more urologically beneficial agents in pharmaceutically effective amounts.

### STATEMENT OF RELATED APPLICATIONS

This application claims the benefit of U.S. Provisional Patent Application Ser. No. 61/008,252, filed Dec. 19, 2007, entitled “Urological Medical Devices For Release Of Urologically Beneficial Agents”, which is incorporated in its entirety by reference herein.

### FIELD OF THE INVENTION

The present invention relates generally to urological medical devices, and more particularly to implantable or insertable urological medical devices which release urologically beneficial agents (also referred to herein as “drugs” and “therapeutic agents”).

### BACKGROUND OF THE INVENTION

Various urological medical devices have been developed for implantation or insertion into patients. As an example, polymeric ureteral stents are widely used to facilitate drainage in the upper urinary tract (e.g., drainage from the kidney to the bladder). They are used, for example, in post endo-urological procedures to act as a scaffold in the event of ureteral obstruction secondary to the procedure. Ureteral stents are also used as palliative devices to provide patency in the presence of congenital defects, strictures or malignancies, as well as in other instances where ureteral obstruction may occur. A schematic illustration of a ureteral stent 10 in accordance with the prior art is illustrated in FIGS. 1A and 1B. The stent 10 has a proximal end 10p and a distal end 10d. It is a tubular polymer extrusion having a shaft 12, a distal renal retention structure (e.g., renal coil or “pigtail” 14), and a proximal retention structure (e.g., bladder coil or “pigtail” 16). These retention structures prevent upward migration of the stent toward the kidney or downward migration of the stent toward the bladder. The shaft 12 in cross-section is a single extruded layer as seen from FIG. 1B, which is taken along line b-b of FIG. 1A. Once properly deployed in the ureter, the stent 10 provides ureteral rigidity and allows the passage of urine. The stent 10 of FIGS. 1A and 1B is further provided with the following features: (a) a tapered tip 11, to aid insertion, (b) multiple side ports 18 (one numbered), which are arranged in a spiral pattern down the length of the body to promote drainage, (c) graduation marks 25 (one illustrated) for visualization by the physician to know when the appropriate length of stent has been inserted into the ureter, and (d) a suture 22, which aids in positioning and withdrawal of the stent. During placement, such ureteral stents 10 are typically placed over a urology guide wire, through a cystoscope and advanced into position. Once the distal end of the stent is advanced into the kidney/renal calyx, the guide wire is removed, allowing the “pigtails” 14, 16 to form in the kidney 19 and bladder 20, as shown in FIG. 2. As shown in FIG. 2, the stent 10 extends through the ureteral orifice 21a and into the bladder 20. For clarity, the ureter entering bladder 20 through the opposite ureteral orifice 21b is not shown.

Ureteral stents are known to be associated with a degree of pain and/or discomfort, particularly in the bladder and flank area after insertion. One way of addressing this pain is to use a softer material, particularly in forming the proximal end of the stent, which engages more sensitive tissue. Stents of this type may employ an extrusion to combine a firm durometer ethylene vinyl acetate copolymer (EVA) at the distal end, which improves stent advancement, and a soft durometer EVA at the proximal end, which improves comfort. A specific example of such a stent is the Polaris™ Dual Durometer Percuflex® Ureteral Stent with HydroPlus™ Coating, available from Boston Scientific, Natick, Mass., USA. Other ways of addressing pain and discomfort include providing systemically administered painkillers or providing devices which release painkillers locally. See, e.g., Pub. No. US 2006/0264912 A1 entitled “Medical devices for treating urological and uterine conditions.”

Another issue associated with ureteral stents is the formation of encrustation in vivo, which may be addressed, for example, through the use of devices that release antimicrobial compounds locally. In this regard, see, e.g., Pub. No. US 2004/0249441 A1 entitled “Implantable or insertable medical device resistant to microbial growth and biofilm formation.”

### SUMMARY OF THE INVENTION

According to an aspect of the present invention, implantable or insertable urological medical devices are provided which release one or more urologically beneficial agents in effective amounts.

Advantages of the present invention are that urological medical devices may be provided which have one or more of the following benefits, among others: (a) relief of pain and/or discomfort associated with the medical device, (b) reduction or elimination of microbial encrustation in vivo, and (c) local release of urologically beneficial agents, thereby avoiding the need for systemic drug administration, which typically requires higher quantities of drug to be efficacious.

Another advantage of the present invention is that urological medical devices may be provided, which act as a delivery platform for essentially any agent a physician or other caregiver may wish to administer.

Another advantage of the present invention is that urological medical devices may be provided, which are initially relatively stiff, improving implantation or insertion, but which become more flexible over time, minimizing pain and/or discomfort after implantation or insertion.

These and other aspects, embodiments and advantages of the present invention will become immediately apparent to those of ordinary skill in the art upon review of the Detailed Description and any claims to follow.

### BRIEF DESCRIPTION OF THE DRAWING

FIG. 1A is a schematic representation of a ureteral stent, according to the prior art. FIG. 1B is a cross-section taken along plane A-A of FIG. 1A.

FIG. 2 shows a ureteral stent like that of FIG. 1 as positioned within the body.

FIG. 3 illustrates several idealized morphologies of polymeric regions comprising a first phase domain based on a first polymer A and a second phase domain based on a second polymer B.

FIG. 4A is a schematic end view of a ureteral stent in accordance with an embodiment of the invention. FIG. 4B is a schematic partial cross-sectional view of the stent of FIG. 4A, taken along plane A-A. FIG. 4C is an expanded view of FIG. 4B corresponding to region B of FIG. 4B.

FIG. 5 is a schematic partial cross-sectional view of the shaft of a ureteral stent in accordance with an embodiment of the present invention

FIG. 6 is a cross-section analogous to that of FIG. 1B. However, FIG. 6 differs from FIG. 1B in that the tubular polymer extrusion comprises a biodisintegrable phase domain (represented by the discontinuous dark regions) and a biostable phase domain (represented by the light region), in accordance with an embodiment of the invention.

FIG. 7 is a cross-section analogous to that of FIG. 1B. However, FIG. 7 differs from FIG. 1B in that the stent comprises a biodisintegrable coating disposed over a biostable tubular polymer extrusion, in accordance with an embodiment of the invention.

FIG. 8 is a schematic partial cross-sectional view of a ureteral stent, in accordance with an embodiment of the invention.

FIG. 9A is a schematic end view of a ureteral stent in accordance with an embodiment of the invention. FIG. 9B is a schematic partial cross-sectional view of the stent of FIG. 9A, taken along plane A-A. FIG. 9C is an expanded view of FIG. 9B corresponding to region B of FIG. 9B.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

In one aspect, the present invention provides implantable or insertable urological medical devices, which are adapted to release one or more urologically beneficial agents in pharmaceutically effective amounts.

For example, in some embodiments, urological medical devices are provided which comprise (a) a biostable composition that comprises a first polymer and a first urologically beneficial agent and (b) a biodisintegrable composition that comprises a second polymer and a second urologically beneficial agent.

In some embodiments, the urological medical devices exhibit an extended release profile for the first urologically beneficial agent and a rapid release profile for the second urologically beneficial agent.

As used herein, a “rapid release profile” is a release profile in which a majority of the urologically beneficial agent is released (e.g., more than 50% is released) shortly after implantation or insertion. For example, a majority of the urologically beneficial agent may be released within 1 day, within 12 hours, within 6 hours, within 3 hours or even within 1 hour of implantation or insertion.

As used herein an “extended release profile” is meant a release profile by which an effective amount of urologically beneficial agent continues to be released at least 7 days after device implantation or insertion, for example after 7 days, after 14 days, after 1 month, after 2 months, or after 3 months or more.

Urological agents may be provided in amounts effective to achieve the relief of pain and/or discomfort associated with the medical device and/or antimicrobial activity, among other beneficial effects. Preferred subjects (also referred to as “patients”) are vertebrate subjects, more preferably mammalian subjects, including human subjects, pets and livestock.

Urological medical devices for use in conjunction with the present invention include any device which is suitable for placement in the urinary tract of a subject, including the kidneys (e.g., in the renal calyx, renal pelvis, etc.), ureters, bladder and urethra. These include various elongated devices including elongated devices having any of a variety of solid and hollow cross-sections including circular (e.g., tubular, multi-lumen, and rod-shaped devices), oval, triangular, and rectangular (e.g., ribbon-shaped devices) cross-sections, among many other regular and irregular cross sections. Specific examples include urological stents, for example, urethral and ureteral stents, and urological catheters (e.g., drainage catheters, guide catheters, etc.).

In some embodiments, devices are provided which are adapted to be advanced over a guide wire and/or advanced through a channel, for example, a channel associated with a guide catheter or scope.

In some embodiments, devices may be employed that take on a particular beneficial shape in vivo, for example, upon removal of a guide wire or upon emergence from a channel (e.g., due to elastic rebound of the material) or upon application of an external stimulus such as heat or light (e.g., where a shape memory material such as a shape memory polymer is employed). For example, the device may take on a non-linear form such as a coiled configuration. Such constructions allow the medical device to be held in place in the urinary tract, for example, by forming a coil or other retention element in the kidney (e.g., in the renal calyx and/or renal pelvis), the bladder, or both.

As indicated above, urologically beneficial agents for use in the medical devices of the invention include antimicrobial agents, agents that reduce pain and/or discomfort (also referred herein as “discomfort reducing agents”), and combinations thereof.

The term “antimicrobial agent” as used herein means a substance that kills microbes and/or inhibits the proliferation and/or growth of microbes, particularly bacteria, fungi and yeast. Antimicrobial agents, therefore, include biocidal agents and biostatic agents as well as agents that possess both biocidal and biostatic properties. In the context of the present invention, the antimicrobial agent kills microbes and/or inhibits the proliferation and/or growth of microbes on and around the surfaces of the implanted or inserted urological medical device, and can therefore inhibit biofilm formation (encrustation) in some cases.

Antimicrobial agents may be selected, for example, from triclosan, chlorhexidine, nitrofurazone, benzalkonium chlorides, silver salts and antibiotics, such as rifampin, gentamicin and minocycline, and combinations thereof, among others.

Further antimicrobial agents may be selected, for example, from suitable members of the following: the penicillins (e.g., penicillin G, methicillin, oxacillin, ampicillin, amoxicillin, ticarcillin, etc.), the cephalosporins (e.g., cephalothin, cefazolin, cefoxitin, cefotaxime, cefaclor, cefoperazone, cefixime, ceftriaxone, cefuroxime, etc.), the carbapenems (e.g., imipenem, metropenem, etc.), the monobactems (e.g., aztreonem, etc.), the carbacephems (e.g., loracarbef, etc.), the glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), among others, as well as combinations and pharmaceutically acceptable salts, esters and other derivatives of the same.

Discomfort reducing agents include antispasmodic agents, alpha-adrenergic blockers, corticosteroids, narcotic analgesic agents, non-narcotic analgesic agents, local anesthetic agents, and combinations thereof.

Antispasmodic agents may be selected, for example, from suitable members of the following: alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, and xenytropium bromide, among others, as well as combinations and pharmaceutically acceptable salts, esters and other derivatives of the same.

Examples of alpha-adrenergic blockers for use in the present invention may be selected from suitable members of the following: alfuzosin, amosulalol, arotinilol, dapiprazole, doxazosin, ergoloid mesylates, fenspiride, idazoxan, indoramin, labetalol, manotepil, naftopidil, nicergoline, prazosin, tamsulosin, terazosin, tolazoline, trimazosin, and yohimbine, among others, as well as combinations and pharmaceutically acceptable salts, esters and other derivatives of the same. Of these, tamsulosin, alfuzosin, doxazosin, prazosin, tamsulosin and terazosin are alpha-1-adrenergic blockers, of which tamsulosin and alfuzosin are selective alpha-1-adrenergic blockers.

Examples of corticosteroids for use in the present invention may be selected from suitable members of the following: betamethasone, cortisone, dexamethasone, deflazacort, hydrocortisone, methylprednisolone, prednisolone, prednisone and triamcinolone, among others, as well as combinations and pharmaceutically acceptable salts, esters and other derivatives of the same.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as combinations and pharmaceutically acceptable salts, esters and other derivatives of the same.

Examples of non-narcotic analgesic agents for use in the present invention may be selected from suitable members of the following: analgesic agents such as acetaminophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as combinations and pharmaceutically acceptable salts, esters and other derivatives of the same.

Examples of local anesthetic agents for use in the present invention may be selected from suitable members of the following: benzocaine, cocaine, lidocaine, mepivacaine, and novacaine, among others, as well as combinations and pharmaceutically acceptable salts, esters and other derivatives of the same.

Many of the above and other urologically beneficial agents may be found, for example, in The Merck Index, 13^{th} Edition, M. J. O'Neil, Senior Editor, published by Merck Research Laboratories, 2001.

In addition to one or more urologically beneficial agents, the urological medical devices of the invention may also contain one or more optional supplemental agents such as imaging agents. For example, x-ray based fluoroscopy is a diagnostic imaging technique that allows real-time patient monitoring of motion within a patient. To be fluoroscopically visible, devices and/or compositions are typically rendered more absorptive of x-rays than the surrounding tissue. In various embodiments of the invention, this is accomplished by the use of radio-opaque agents. Examples of radio-opaque agents for use in connection with x-ray fluoroscopy include metals, metal salts and oxides (particularly bismuth salts and oxides), and iodinated compounds, among others. More specific examples of such radio-opaque agents include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine, among others.

In certain embodiments of the invention, one or more urologically beneficial agents are disposed within a polymeric composition.

As used herein, a “polymeric” composition is a composition (e.g., a device component such as device body or a device coating, a phase domain, etc.) one that contains one or more polymers, for example, 50 wt % or less to 75 wt % to 90 wt % to 95 wt % to 97.5 wt % to 99 wt % polymers, or more.

In some embodiments, a polymeric component (e.g., a device body, device coating, etc.) may comprise two or more immiscible polymers, in which case the polymeric component will comprise two or more distinct phase domains. Phase domains can be visualized by various techniques known in the polymer art, including microscopic techniques such as optical microscopy, AFM (atomic force microscopy), TEM (transition electron microscopy) or SEM (scanning electron microscopy), after staining with a suitable stain if desired.

As used herein, “polymers” are molecules containing multiple copies (e.g., from 2 to 5 to 10 to 100 to 1000 to 10,000 to 100,000 or more copies) of one or more constitutional units, commonly referred to as monomers. As used herein, the term “monomers” may refer to the free monomers and those that are incorporated into polymers, with the distinction being clear from the context in which the term is used. Polymers may take on a number of configurations, which may be selected, for example, from cyclic, linear, branched and networked (e.g., crosslinked) configurations. Branched configurations include star-shaped configurations (e.g., configurations in which three or more chains emanate from a single branch point, for instance an initiator molecule or a linking molecule), comb configurations (e.g., configurations having a main chain and a plurality of side chains), dendritic configurations (e.g., arborescent and hyperbranched polymers), and so forth. As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. As used herein, “block copolymers” are copolymers that contain two or more polymer blocks that differ in composition, for instance, because a constitutional unit (i.e., monomer) is found in one polymer block that is not found in another polymer block. As used herein, a “polymer block” is a grouping of constitutional units (e.g., 2 to 5 to 10 to 25 to 50 to 100 to 250 to 500 to 1000 or more units). Blocks can be branched or unbranched, and they may be networked (e.g., by crosslinking). Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution.

As used herein, a “biodissolvable” polymer is one that is soluble (e.g., having a solubility of at least 0.01 g/ml in urine at body temperature (which may be determined in artificial urine at body temperature).

As used herein, a “biodisintegrable” composition is one that undergoes significant (i.e., at least 50 wt % up to and including total disappearance) disintegration (e.g., due to dissolution, degradation, etc.) within a period of 7 days after implantation or insertion in the urinary tract as a result of the normal flow of urine, for example, within 7 days, within 5 days, within 3 days or within 1 day or less.

As used herein, a “biostable” composition is one that remains substantially intact (i.e., loss of weight less than 50 wt %) in the urinary tract over the maximum time period for which the medical device is approved to reside in the body. For example, in the case of a biostable ureteral stent body, the stent body may remain substantially intact in vivo for a period of at least 90 days.

In some embodiments of the invention, urological medical devices are provided which comprise (a) a biostable composition that comprises a first polymer and a first urologically beneficial agent and (b) a biodisintegrable composition that comprises a second polymer and a second urologically beneficial agent. In these embodiments, the first and second polymers typically differ from one another (e.g., the second polymer may be biodissolvable whereas the first polymer may not be biodissolvable). In these embodiments, the first and second urologically beneficial agents may be the same or different. Due to the nature of the compositions (one biodisintegrable and one biostable), the second urologically beneficial agent is released from the device at a rate that is substantially greater than the first urologically beneficial agent.

Examples of polymers for use in the biodisintegrable compositions of the invention include suitable members of the following, among others: polysaccharides including celluloses, for example, ionic celluloses such as sodium carboxymethyl cellulose, and non-ionic celluloses, for example, hydroxyalkyl celluloses such as hydroxymethyl cellulose, hydroxyethyl cellulose, and hydroxyproyl cellulose (e.g., Klucel® G and Klucel® E), further polysaccharides including alginic acid, pectinic acid, hyaluronic acid, dextran, carboxymethyl dextran, modified dextran, starch, carboxymethyl starch, and additional polymers including polyvinyl alcohol, polyethylene glycol, polyethylene terephthalate glycol (PETG), polyalkylene oxides including polyethylene oxide and polypropylene oxide, poly(acrylic acid), poly(methacrylic acid), polyvinylpyrrolidone, polyacrylamide, poly(N-alkylacrylamides), poly(vinyl sulfonic acid), and polypeptides (e.g., polyglutamic acid, polylysine, etc.), as well as salts, copolymers and blends of the forgoing.

Polymers for use in the biostable compositions of the invention may be selected, for example, from alkene polymers, polycarbonates, silicone polymers, polyurethanes, and poly(ether-block-amides), among others.

Alkene polymers include polyalkene homopolymers as well as copolymers with themselves and with various other monomers including those selected from vinyl aromatic monomers such as styrene and alpha-methyl styrene, acrylic acid, methacrylic acid, and vinyl acetate. Examples of alkene monomers include ethylene, propylene, isobutylene, 1-butene, 1-pentene, 4-methyl-1-pentene, dienes such as 1,3-butadiene, 2-methyl-1,3-butadiene (isoprene), 2,3-dimethyl-1,3-butadiene, 2-ethyl-1,3-butadiene, 1,3-pentadiene, 2-methyl-1,3-pentadiene, 4-butyl-1,3-pentadiene, 2,3-dibutyl-1,3-pentadiene, 2-ethyl-1,3-pentadiene, 1,3-hexadiene, 1,3-octadiene, and 3-butyl-1,3-octadiene, among others.

Specific examples of alkene copolymers include poly(ethylene-co-vinyl acetate) (EVA), poly(ethylene-co-methacrylic acid), poly(ethylene-co-acrylic acid), and poly(isobutylene-co-styrene), among many others. Among EVA copolymers are included random and other copolymers having a vinyl acetate weight percent ratio of from about 0.5% to 1% to 2% to 5% to 15% to 20% to 30% to 40% or more. In general, the higher the vinyl acetate content, the lower the stiffness and Durometer of the EVA. Thus, the stiffness and durometer may be varied within the device, in certain embodiments. Taking a ureteral stent as an example, a stent may be produced having distinct end regions of different durometer value with a transitional region in between.

Polycarbonates are derived from the reaction of carbonic acid derivatives with aromatic, aliphatic, or mixed diols. They may be produced, for example, by the reaction of phosgene with a diol in the presence of an appropriate hydrogen chloride receptor or by a melt transesterification reaction between a diol and a carbonate ester. Polycarbonates can be made from a wide variety of starting materials. For example, a common polycarbonate, bisphenol A polycarbonate, is a polycarbonate made by reacting bisphenol A with phosgene by condensation. For further information, see, e.g., U.S. Pat. No. 5,580,924 and the references cited therein.

Silicone polymers (also referred to as polysiloxanes) are polymers comprising one or more types of siloxane units,

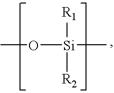

where R₁ and R₂ can be the same or different and may be selected from linear, branched and cyclic alkyl groups, aromatic groups and alky-aromatic groups, for example, having from 1 to 10 carbon atoms. Examples include polydimethylsiloxane, polydiethylsiloxane, polymethylethylsiloxane, polymethylphenylsiloxane, and polydiphenylsiloxane, among many others.

In general, polyurethanes are a family of polymers that are synthesized from polyfunctional isocyanates (e.g., diisocyanates, including both aliphatic and aromatic diisocyanates) and polyols (also, referred to as macroglycols, e.g., macrodiols). Commonly employed macroglycols include polyester glycols, polyether glycols and polycarbonate glycols. Typically, aliphatic or aromatic diols are also employed as chain extenders, for example, to impart the useful physical properties described above. Examples of diol chain extenders include butane diol, pentane diol, hexane diol, heptane diol, benzene dimethanol, hydraquinone diethanol and ethylene glycol. Polyurethanes are commonly classified based on the type of macroglycol employed, with those containing polyester glycols being referred to as polyester polyurethanes, those containing polyether glycols being referred to as polyether polyurethanes, and those containing polycarbonate glycols being referred to as polycarbonate polyurethanes. Polyurethanes are also commonlydesignated aromatic or aliphatic on the basis of the chemical nature of the diisocyanate component in their formulation. For example, U.S. Patent App. No. 2004/0131863 to Belliveau et al. describes aliphatic polycarbonate polyurethanes which are the reaction products of (a) a hydroxyl terminated polycarbonate, (b) an aliphatic diisocyanate and (c) a lower aliphatic chain extender. Hydroxyl terminated polycarbonate polyol may be prepared by reacting a glycol with a carbonate, as disclosed in U.S. Pat. No. 4,131,731. Suitable aliphatic diisocyanates include hexamethylene diisocyanate (HDI), isophorone diisocyanate (IPDI), trimethyl hexamethylene diisocyanate (TMHDI), dicyclohexyl methane diisocyanate (HMDI), and dimer acid diisocyanate (DDI), with HMDI said to be preferred. Suitable chain extenders include lower aliphatic glycols having from about 2 to about 10 carbon atoms, such as, for instance ethylene glycol, diethylene glycol, propylene glycol, dipropylene glycol, 1,4-butanediol, 1,6-hexanediol, 1,3-butanediol, 1,5-pentanediol, 1,4-cyclohexanedimethanol hydroquinone di(hydroxyethyl) ether, neopentyglycol, and the like, with 1,4-butanediol said to be preferred.

Another group of polymers are block copolymers comprising polyether blocks (i.e., polymer blocks containing multiple C—O—C linkages) and polyamide blocks (i.e., polymer blocks containing multiple —NH—CO— linkages), sometimes referred to as poly(ether-b-amides) or polyether-block-amides. A few specific examples of polyether blocks include homopolymeric and copolymeric blocks of the formulas (a)—[R₁—O—]ₙ— or (b)—[R₁—O—R₂—O]ₙ—, where R₁ and R₂ can be the same or different and may be selected from linear, branched and cyclic alkyl groups, aromatic groups and alky-aromatic groups, for example, having from 1 to 10 carbon atoms (more typically linear or branched alkyl groups having from 1 to 6 carbons) and where n is an integer of 5 or more, typically 10 to 100 to 1000 to 10,000 or more. Polyethers may be formed, for example, from ring opening addition polymerization of cyclic ethers. Examples include polyethylene oxide, where R₁═R₂=dimethylene (i.e., [—(CH₂)₂—O—]ₙ), which is commonly referred to as polyethylene glycol or as polyethylene oxide), trimethylene oxide, where R₁═R₂=trimethylene (i.e., [—(CH₂)₃—O—]ₙ), polypropylene oxide, where R₁═R₂=methyl substituted dimethylene (i.e., [—CH₂CH₂(CH₃)—O—]ₙ, referred to as polypropylene glycol or polypropylene oxide), and polytetrahydrofuran, where R₁═R₂=tetramethylene (i.e., —[(CH₂)₄—O]—ₙ, which is referred to as polytetramethylene glycol, polytetramethylene oxide (PTMO), or terathane). Examples of polyamide blocks, which may be provided, for example, as homopolymeric or copolymeric blocks, include polyamides of the formula —[R₃—NH—CO]ₘ— or —[NH—R₃—NH—CO—R₄—CO]ₘ—, where R₃ and R₄ can be the same or different and may be selected from linear, branched and cyclic alkyl groups, aromatic groups and alky-aromatic groups, for example, of 1 to 20 carbon atoms (more typically linear or branched alkyl groups having from 1 to 15 carbons, such as methyl, ethyl, propyl, isopropyl, and so forth) and where m is an integer of 5 or more, typically 10 to 100 to 1000 to 10,000 or more. Specific examples include nylons, such as nylon 6, nylon 4/6, nylon 6/6, nylon 6/10, nylon 6/12, nylon 11 and nylon 12. A specific example of a polyether-polyamide block copolymer is poly(tetramethylene oxide)-b-polyamide-12 copolymer, available from Elf Atochem as PEBAX.

A wide range of agent loadings (e.g., selected from urologically beneficial agents and optional supplemental agents such as radio-opaque agents, etc.) may be used in conjunction with the urological medical devices of the present invention, with the effective amount being readily determined by those of ordinary skill in the art. Typical loadings range, for example, from than 1 wt % or less to 2 wt % to 5 wt % to 10 wt % to 25 wt % to 50 wt % or more, for the various biodisintegrable and biostable compositions of the invention.

As noted above, in one aspect of the invention, urological medical devices are provided which comprise (a) a biostable composition that comprises a first polymer and a first urologically beneficial agent and (b) a biodisintegrable composition that comprises a second polymer and a second urologically beneficial agent. In these embodiments, the first and second polymers typically differ from one another, whereas the first and second urologically beneficial agents may be the same or different. Due to the nature of the compositions (one biodisintegrable, one biostable), the second urologically beneficial agent is released from the device body at a rate that is substantially greater than the first urologically beneficial agent. For example, in some embodimets, the second urologically beneficial agent may be released in a rapid release profile, whereas first urologically beneficial agent may be released in an extended release profile.

As an example, the first polymer may be a non-biodissolvable polymer such as EVA, while the second polymer may be a biodissolvable polymer such as a Klucel® polymer (e.g., Klucel® EF or Klucel® HF, among others). The first urologically beneficial agent may be, for instance, an antimicrobial agent (e.g., triclosan), which is relatively slowly released, while the second urologically beneficial agent may be, for instance, a discomfort reducing agent (e.g., an NSAID such as aspirin or ketorolac), which is relatively rapidly released. As another example, the second urologically beneficial agent may be, for instance, a discomfort reducing agent (e.g., a powerful pain killer, such as a narcotic pain killer), which is relatively rapidly released, and the first urologically beneficial agent may be a different discomfort reducing agent (e.g., a less powerful painkiller, such as aspirin or ketorolac), which is relatively slowly released.

In some embodiments of the invention, the biostable composition corresponds to biostable phase domain that comprises the first polymer and the first urologically beneficial agent, whereas the biodisintegrable composition corresponds to a biodisintegrable phase domain that comprises the second polymer and the second urologically beneficial agent. For instance, urological medical devices may be provided which comprise a body portion (e.g., a catheter body, stent body, etc.) that comprises such biodisintegrable and biostable phase domains.

Some typical idealized morphologies of polymeric regions comprising a first phase domain based on a second polymer A and a second phase domain based on a first polymer B are illustrated in FIG. 3. As the fraction of A goes from high to low, morphologies that may be encountered are: (a) spheres of B in a matrix of A, (b) cylinders of B in a matrix of A, (c) dual labyrinths of B in a matrix of A (e.g., double gyroid), (d) alternating sheets of A and B, (e) dual labyrinths of A in a matrix of B, (f) cylinders of A in a matrix of B, and (g) spheres of A in a matrix of B.

Preferably, the biostable phase domain will be a continuous phase domain (e.g., idealized morphologies e, f and g in FIG. 3, where A is the biodisintegrable phase and where B is the biostable phase) ensuring that the device body remains substantially intact. In certain embodiments, a bicontinuous phase distribution may be preferred as this will allow effective disintegration of the biodisintegrable phase domain while at the same time ensuring that the device body remains substantially intact.

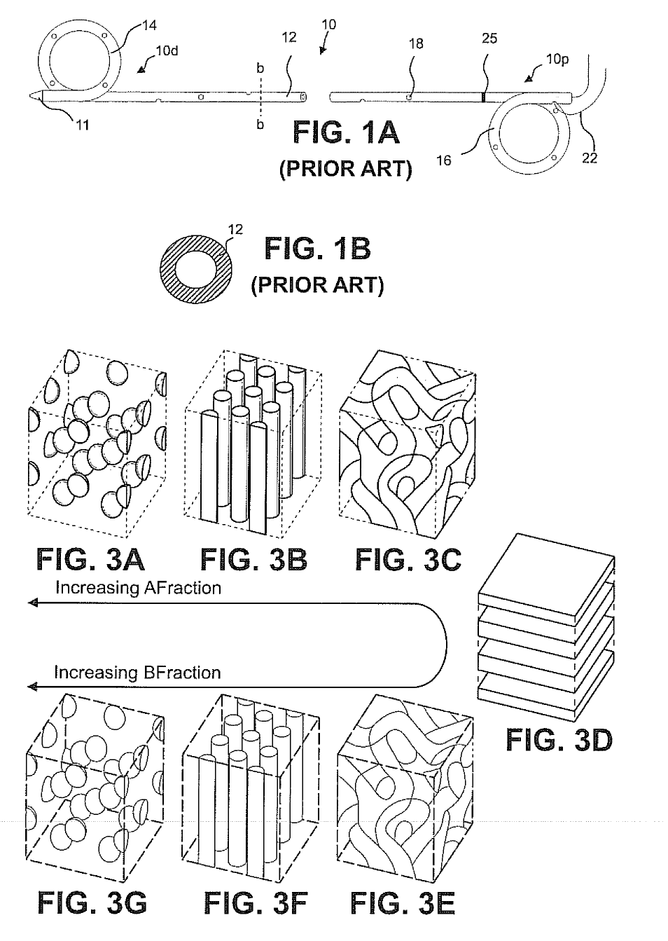

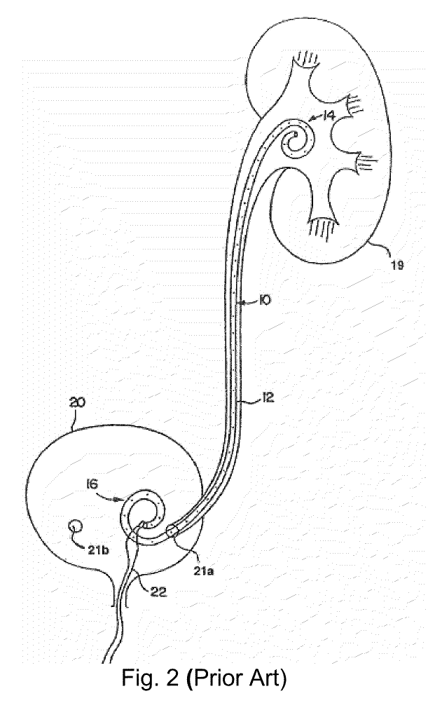

As an example, a ureteral stent having a design analogous to that of FIGS. 1A-1B may be formed. In this design, however, and with reference to FIG. 6, the tubular polymer extrusion 112 comprises (a) a biostable phase domain (schematically represented by the continuous light region) that comprises a first polymer and a first urologically beneficial agent and (b) a biodisintegrable phase domain (schematically represented by the discontinuous dark regions) that comprises a second polymer and a second urologically beneficial agent.

As previously indicated, in one aspect of the invention, urological medical devices are provided which comprise (a) a biostable composition that comprises a first polymer and a first urologically beneficial agent and (b) a biodisintegrable composition that comprises a second polymer and a second urologically beneficial agent.

In some embodiments of the invention, the biostable composition corresponds to a biostable device component that comprises the first polymer and the first urologically beneficial agent and the biodisintegrable composition corresponds to a biodisintegrable device component that comprises the second polymer and the second urologically beneficial agent.

For instance, the biostable device component may correspond to a medical device body and the biodisintegrable device component may correspond to a coating or cladding layer on the medical device body.

As a specific example, a ureteral stent having a design analogous to that of FIGS. 1A-1B may be formed. In the present design, however, and with reference to the cross-sectional view of FIG. 7, the stent comprises a biostable tubular polymer extrusion 112 which comprises a first polymer (e.g., a non-biodissolvable polymer such as EVA, etc.) and a first urologically beneficial agent (e.g., an antimicrobial agent such as triclosan, a discomfort reducing agent, etc.). The stent further comprises a biodisintegrable coating 126 comprising a second polymer (e.g., a biodissolvable polymer such as Klucel®, etc.) and a second urologically beneficial agent (e.g., a discomfort reducing agent, etc.), disposed over the biostable tubular polymer extrusion 112. As with all the examples given herein, various other combinations of polymers and agents may be selected, for example, from those described above, among others. In certain embodiments the biodisintegrable coating 126 is a relatively stiff material and is of a thickness sufficient to significantly increase (e.g., by 25%, 50% or more) the stiffness of the device.

To the extent that the biodisintegrable component is a relatively stiff material, an advantage of an embodiment like that of FIG. 7 (as well as various embodiments described below) is that urological medical devices may be provided, which are initially relatively stiff, enhancing implantation or insertion, but which become more flexible over time, thereby reducing pain and/or discomfort after implantation or insertion. This feature also allows the use materials for the biostable component which are softer than otherwise would be practical.

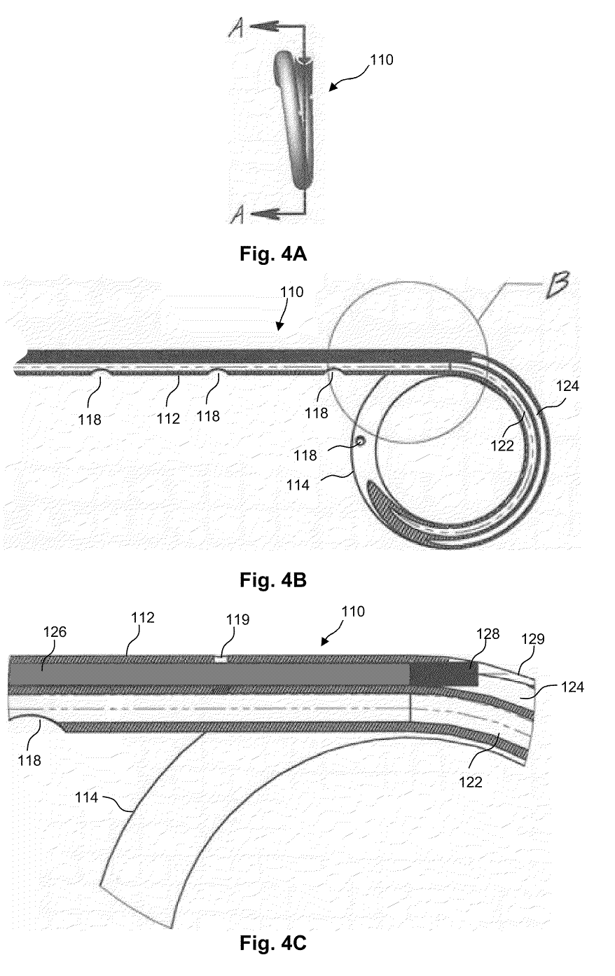

As another example, turning now to FIG. 4A, there is shown an end view of a ureteral stent 110 in accordance with an embodiment of the invention. FIG. 4B is a partial cross-sectional view of the stent 110 of FIG. 4A, taken along plane A-A. FIG. 4C is an expanded view of FIG. 4B corresponding to region B of FIG. 4B. FIGS. 4A-4C illustrate the renal coil 114 of the shaft and a more linear portion of the shaft 112 extending in the direction of the bladder coil (not shown) of the stent 110. The shaft 112 is a biostable device component that comprises a first polymer (e.g., a non-biodissolvable polymer such as EVA, etc.) and a first urologically beneficial agent (e.g., an antimicrobial agent such as triclosan, etc.). The shaft 112 is a dual lumen design, comprising a drainage lumen 122 with drainage ports 118 (i.e., openings extending from the drainage lumen 122 to the exterior of the device) to promote drainage, and a drug delivery lumen 124 with one or more drug delivery ports 119 (i.e., openings extending from the drug delivery lumen 124 to the exterior of the device) to promote drug delivery. Within the drug delivery lumen 124 is disposed a biodisintegrable device component 126 that comprises a second polymer (e.g., a biodissolvable polymer such as Klucel®, etc.) and a second urologically beneficial agent (e.g., a discomfort reducing agent, etc.), which is in solid cylindrical form (e.g., in the form of a “drug stick”). In the embodiment shown, aperture 129 provides access to the drug delivery lumen 124 into which the biodisintegrable device component 126 has been introduced. Also shown is a plug 128, which can be inserted to block the lumen 124 at a position adjacent to the aperture 129, after the biodisintegrable device component 126 has been introduced. This allows, for example, for the biodisintegrable device component 126 to be inserted by a device manufacturer or by health care provider. In the latter case, the health care provider is provided with essentially unlimited flexibility as to the nature of the second urologically beneficial agent. Upon introduction of the stent 110 into the body of a patient, the drainage lumen 122 (with ports drainage ports 118) acts to promote drainage of urine through the ureter, whereas the drug delivery lumen 124 (with drug delivery ports 119) acts to promote drug delivery. For example, urine can enter the drug delivery lumen 124 via drug delivery ports 119, which urine acts to dissolve the biodisintegrable device component 126. The resulting urine (which comprises the second urologically beneficial agent) is then available for transport from the drug delivery lumen 124 to the patient via drug delivery ports 119. At the same time, the first urologically beneficial agent is released from the shaft into urine that contacts the shaft as well.

As indicated above, in some embodiments, the biodisintegrable device component 126 provides increased stiffness, facilitating the initial placement of the device 100, whereas the biostable shaft 112 is formed from a softer base material that provides for longer term in-dwelling patient comfort subsequent to biodistintegration of the component 126.

A similar device is schematically illustrated in FIG. 8, which shows a partial cross-sectional view of a ureteral stent 110 in accordance with an embodiment of the invention. As in FIGS. 4A-4C, FIG. 8 illustrates the renal coil 114 of the shaft 112 and a portion of the shaft 112 extending in the direction of the bladder coil (not shown) of the stent 110. The shaft 112 is a biostable device component that comprises a first polymer (e.g., a non-biodissolvable polymer such as EVA, etc.) and a first urologically beneficial agent (e.g., an antimicrobial agent such as triclosan, etc.). The shaft 112 is a dual lumen design, comprising a drainage lumen 122 with drainage ports 118 to promote drainage, and a drug delivery lumen 124 with drug delivery ports 119 to promote drug delivery. Within the drug delivery lumen 124 is disposed a solid cylindrical biodisintegrable device component 126 (e.g., a “drug stick”), which comprises a second polymer (e.g., a biodissolvable polymer such as Klucel®, etc.) and a second urologically beneficial agent (e.g., a discomfort reducing agent, etc.). The cross-section for the biodisintegrable device component 126 is taken at a position more proximal (bladder end) than the cross-section taken for the shaft 112 as shown. The biodisintegrable device component 126 may be introduced into the drug delivery lumen 124 from either end of the stent 110. As an alternative, the biodisintegrable device component 126 may be introduced via an aperture (not shown) which may be provided with a plug (not shown) as in FIGS. 4A-4C.

Biodisintegrable device components in forms other than solid cylindrical forms can also be disposed in the urological medical devices of the invention, including components in the form of hollow cylinders or particles (e.g., microspheres), among others.

In certain embodiments, the microspheres may contain, for example, a structural polymer selected from polyvinyl alcohol, polyacrylamide, polyethylene glycol, polyamides, polyureas, polyurethanes, and derivatives thereof, among others. Other examples, of structural polymers include polysaccharides such as hydroxyalkyl celluloses, among others. Processes of manufacturing polymeric microspheres include processes such as those set forth in Pub. No. US 2003/0183962 to Buiser et al., among others. In this process, beads of a predetermined size are formed from a starting material which may include a template polymer. Examples of template polymers include alginate, polysaccharide, carrageenan, chitosan, and hyaluronic acid, and carboxylic-, sulfate-, or amine-functionalized polymers, among others. Subsequently, the beads are contacted a structural polymer such as one of those listed above. After crosslinking of the structural polymer using a suitable crosslinking agent has taken place, the template polymer may be removed to form the finished microspheres. Examples of crosslinking agents include formaldehyde or glutaraldehyde, among many others. The structural polymer may also be crosslinked by application of photoinitiation, an ionic agent, or actinic radiation, such as ultraviolet, or gamma radiation, or an electron beam. The microspheres may be loaded with therapeutic agent during their formation. For example, polymer may be dissolved in a solvent, along with a desired drug. The spheres may then be formed by crosslinking using one or more of the methods listed above. Alternatively, the microspheres may be loaded with therapeutic agent after their formation.

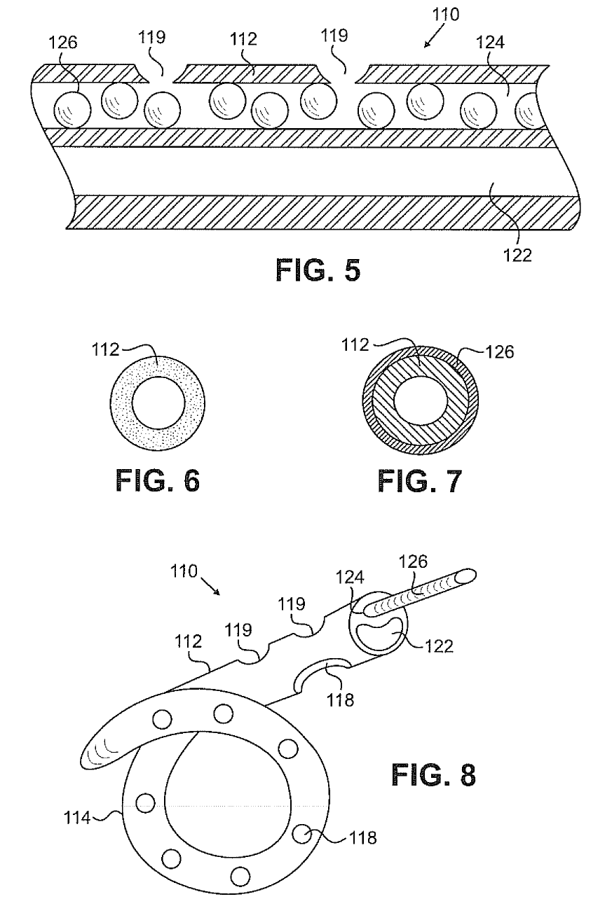

FIG. 5 is a schematic partial cross-sectional view of the shaft 112 of a ureteral stent 110 in accordance with the present invention. As in FIGS. 4A-4C, the shaft 112 is a dual lumen design, comprising a drainage lumen 122 with ports (not shown) to promote drainage, and a drug delivery lumen 124 with drug delivery ports 119 to promote drug delivery. The shaft 112 is a biostable device component that comprises a first polymer (e.g., a non-biodissolvable polymer such as EVA, etc.) and a first urologically beneficial agent (e.g., an antimicrobial agent such as triclosan, etc.). Within the drug delivery lumen 124 are disposed a plurality of solid spheres 126, which are biodisintegrable device components that comprise a second polymer (e.g., a biodissolvable polymer such as Klucel®, etc.) and a second urologically beneficial agent (e.g., a discomfort reducing agent, etc.). As with FIGS. 4A-4C, the drug delivery lumen 124 of the shaft 112 may be provided with an aperture and plug (not shown), which can be used to load and retain the spheres 126. Upon introduction of the stent 110 into the body of a patient, the drainage lumen 122 (with its associated drainage ports) acts to promote drainage of urine through the ureter, while the drug delivery lumen 124 (with its associated drug delivery ports 119) acts to promote delivery of the urologically beneficial agent.

In the above illustrations, the biodisintegrable device components are shown positioned in the lumen of the medical device. In other embodiments of the invention, a kit is provided, which comprises: (a) a urological medical device comprising a biostable elongate body having at least one lumen (e.g., a dual lumen ureteral stent body with a drug delivery lumen and a drainage lumen) and (b) at least one solid biodisintegrable device component (e.g., drug containing spheres, rods, etc.) that is sized to fit into at least one lumen of the biostable elongate body (e.g., the drug delivery lumen of a dual lumen ureteral stent body). As above, the biostable elongate body comprises a first polymer (e.g., a non-biodissolvable polymer such as EVA, etc.) and a first urologically beneficial agent (e.g., an antimicrobial agent such as triclosan, etc.) and the biodisintegrable device component comprises a second polymer (e.g., a biodissolvable polymer such as Klucel®, etc.) and a second urologically beneficial agent (e.g., a discomfort reducing agent, etc.).

According to another aspect of the invention, a urological medical device is provided that comprises a medical device body, which has a first lumen and is formed of a biostable composition that comprises a polymer (e.g., a non-biodissolvable polymer such as EVA, etc.) and a urologically beneficial agent (e.g., an antimicrobial agent such as triclosan, etc.). The first lumen comprises a biostable or biodisintegrable porous film material.

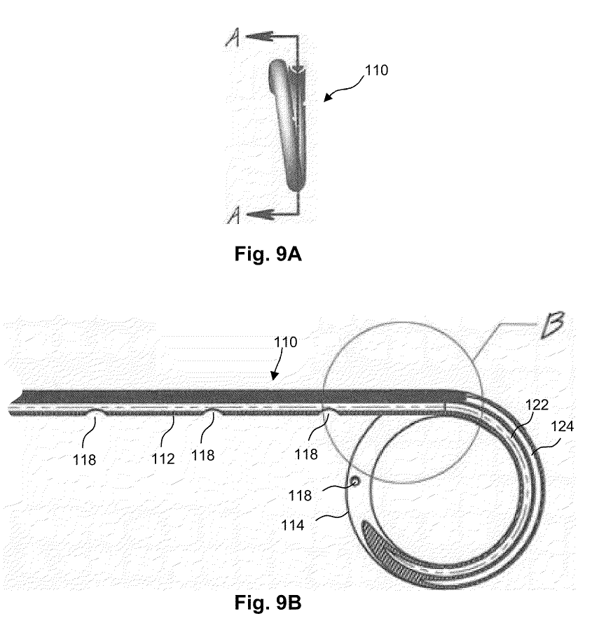

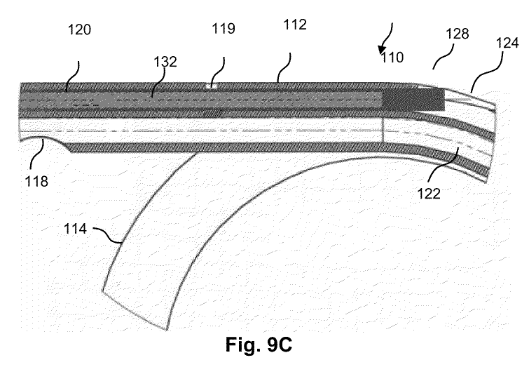

For example, turning now to FIG. 9A, there is shown a schematic end view of a ureteral stent 110 in accordance with an embodiment of the invention. FIG. 9B is a partial cross-sectional view of the stent 110 of FIG. 9A, taken along plane A-A. FIG. 9C is an expanded view corresponding to region B in FIG. 9B. FIGS. 9A-9C illustrate the renal coil 114 of the shaft and a portion of the shaft 112 extending in the direction of the bladder coil (not shown) of the stent 110. The shaft 112 is a dual lumen design, comprising a drainage lumen 122 with drainage ports 118 to promote urine drainage, and a drug delivery lumen 124 with drug delivery ports 119 to promote drug delivery. The shaft 112 is a biostable device component that comprises a polymer (e.g., a non-biodissolvable polymer such as EVA, etc.) and a first urologically beneficial agent (e.g., an antimicrobial agent, etc.). The drug delivery lumen 124 of the shaft 112 is lined with a porous material 120 which may be biostable or biodisintegrable. In a specific example, the porous material may be a porous material like those described in U.S. Pat. No. 5,282,785 to Shapland et al., U.S. Pat. No. 5,569,198 to Racchini, or U.S. Pat. No. 5,458,568 to Racchini et al. The drug delivery lumen 124 of the shaft 112 is provided with a removable plug 128 (or a septum) which allows a liquid solution or dispersion of a second urologically beneficial agent 132 (e.g., a solution of a discomfort reducing agent, etc.) to be introduced into the drug delivery lumen 124 and at least partially absorbed by the layer of porous material 120. The first and second urologically beneficial agents may be the same or different. Upon introduction of the stent 110 into the body of a patient, the drainage lumen 122 (with ports drainage ports 118) acts to promote drainage of urine through the ureter, whereas the drug delivery lumen 124 (with drug delivery ports 119) acts to promote drug delivery of the second urologically beneficial agent. For example, urine can enter the drug delivery lumen 124 via drug delivery ports 119, which urine takes up the second urologically beneficial agent and is then transported from the drug delivery lumen 124 to the patient via drug delivery ports 119. At the same time, the first urologically beneficial agent is released from the shaft into urine that contacts the shaft as well. In some embodiments, the stent 110 exhibits an extended release profile for the first urologically beneficial agent and a rapid release profile for the second urologically beneficial agent.

Numerous techniques are available for forming device components (up to and including entire devices) in accordance with the present invention.

For example, where the device components is formed from one or more polymers having thermoplastic characteristics, a variety of standard thermoplastic processing techniques may be used to form the device components, including injection molding, compression molding, blow molding, spinning, vacuum forming and calendaring, extrusion into sheets, fibers, rods, tubes and other cross-sectional profiles of various lengths, and combinations of these processes. Using these and other thermoplastic processing techniques, a wide variety of device components can be formed.

Other processing techniques besides thermoplastic processing techniques may also be used to form the polymeric device components of the present invention, including solvent-based techniques. Using these techniques, a polymeric device component can be formed by (a) first providing a solution or dispersion that contains (i) solvent, (ii) polymer(s), (iii) urologically beneficial agent(s) (in certain embodiments), and (iv) any optional supplemental agent(s), and (b) subsequently removing the solvent. The solvent that is ultimately selected will contain one or more solvent species (e.g., water and/or one or more organic solvents), which are generally selected based on their ability to dissolve the polymer(s) that form the polymeric device component (and in certain embodiments, the urologically beneficial agent(s) and any optional supplemental agent(s)), in addition to other factors, including drying rate, surface tension, etc. Preferred solvent-based techniques include solvent casting techniques, spin coating techniques, web coating techniques, solvent spraying techniques, dipping techniques, techniques involving coating via mechanical suspension including air suspension, ink jet techniques, electrostatic techniques, and combinations of these processes, among others.

In certain embodiments of the invention, a polymer melt (where thermoplastic processing is employed) or a polymer-containing solution (where solvent-based processing is employed) is applied to a substrate to form a polymeric device component, which melt or solution may also contain urologically beneficial agent(s) and/or any optional supplemental agent(s). For example, the substrate can correspond to all or a portion of an implantable or insertable urological medical device body to which a polymeric coating is applied. The substrate can also be, for example, a template, such as a mold, from which the polymeric device component is removed after solidification. In certain other embodiments, for example, extrusion and co-extrusion techniques, one or more polymeric device components are formed without the aid of a substrate.

In a more specific example, an entire stent body may be extruded as a device component. In another, a biodisintegrable layer may be co-extruded along with an underlying biostable stent body. In another, a biodisintegrable layer may be provided by spraying or extruding a coating layer onto a pre-existing biostable stent body. In yet another more specific example, an entire stent body may be cast in a mold. In still another more specific example, a biodisintegrable device component may be extruded or cast in a size that is suitable for insertion into a drug delivery lumen of a pre-existing biostable stent body.

As seen from the above, where various agents—for example, urologically beneficial agent(s) and/or any optional supplemental agent(s)—are stable under the polymer processing conditions employed, then they can be combined with the polymer(s) and co-processed along with the same to form the polymeric device component of interest. Alternatively, the agent or agents of choice can be introduced subsequent to the formation of the polymeric component using techniques such as imbibing (e.g., where the agent or agents of choice are dissolved or dispersed in a solvent and then contacted with the device component, for instance, by spraying, dipping, etc.).

In some embodiments, mixing or compounding of at least one polymer and at least one additional agent (e.g., selected from urologically beneficial agents and optional supplemental agents such as imaging agents) may be performed using any suitable processing technique known in the art. For example, where thermoplastic materials are employed, a polymer melt may be formed. A common way of doing so is to apply mechanical shear to a mixture of the polymer(s) and the agent(s). After compounding, the material may be processed using, for example, one or more of the thermoplastic techniques described above, among others. Materials may also be compounded, for example, by dissolving them in a common solvent followed by solvent evaporation.

Porous materials may be produced using various methods known in the art. For example, porous materials may be formed using phase inversion processes, track-etch processes or laser ablation, as described in U.S. Pat. No. 5,282,785 to Shapland et al.

Various aspects of the invention of the invention relating to the above are enumerated in the following paragraphs:

Aspect 1. An implantable or insertable urological medical device comprising (a) a biostable device body comprising a first polymer and a first urologically beneficial agent, the device body comprising a first lumen, and (b) a biodisintegrable component comprising a second polymer and a second urologically beneficial agent disposed within the first lumen, wherein the first and second polymers differ from one another, wherein the first and second urologically beneficial agents may be the same or different, and wherein upon implantation or insertion in a subject, the first urologically beneficial agent exhibits an extended release profile and the second urologically beneficial agent exhibits a rapid release profile.

Aspect 2. The urological medical device of aspect 1, wherein the first and second urologically beneficial agents are different.

Aspect 3. The urological medical device of aspect 2, wherein the first urologically beneficial agent is an antimicrobial agent or a discomfort reducing agent, and wherein the second urologically beneficial agent is a discomfort reducing agent.

Aspect 4. The urological medical device of aspect 1, wherein the first polymer is EVA and the second polymer is a biodissolvable polymer.

Aspect 5. The urological medical device of aspect 1, wherein the first polymer is EVA and the second polymer is a biodissolvable cellulose.

Aspect 6. The urological medical device of aspect 5, wherein biodissolvable cellulose is selected from hydroxymethyl cellulose, hydroxyethyl cellulose, hydroxyproyl cellulose, and combinations thereof.

Aspect 7. The urological medical device of aspect 1, wherein the biodisintegrable component is in the form of an elongated rod.

Aspect 8. The urological medical device of aspect 1, comprising a plurality of biodisintegrable components.

Aspect 9. The urological medical device of aspect 8, wherein the biodisintegrable components are in the form of spheres.

Aspect 10. The urological medical device of aspect 1, further comprising an aperture extending from the first lumen to the exterior of the device, the aperture being sized to allow the introduction of the biodisintegrable component from the exterior of the device into the first lumen, and a plug that is sized to plug the aperture.

Aspect 11. The urological medical device of aspect 1, further comprising a plurality of first openings extending between the exterior of the device and the first lumen.

Aspect 12. The urological medical device of aspect 1, wherein the urological medical device is a ureteral stent and wherein the first lumen extends along at least a portion of the length of the ureteral stent.

Aspect 13. The urological medical device of aspect 12, wherein the ureteral stent comprises a second lumen extending along at least a portion of the length of the ureteral stent.

Aspect 14. The urological medical device of aspect 13, wherein the ureteral stent comprises a plurality of first openings that extend from the exterior of the stent to the first lumen and a plurality of second openings that extend from the exterior of the stent to the second lumen.

Aspect 15. A kit comprising (a) implantable or insertable urological medical device comprising a biostable device body that comprises a first polymer and a first urologically beneficial agent, the device body comprising a first lumen and an aperture between the first lumen and the exterior of the device and (b) a biodisintegrable component comprising a second polymer and a second urologically beneficial agent sized to fit through the aperture and into the first lumen, wherein the first and second polymers differ from one another, wherein the first and second urologically beneficial agents may be the same or different, and wherein upon implantation or insertion into a subject, the first urologically beneficial agent exhibits an extended release profile and the second urologically beneficial agent exhibits a rapid release profile.

Aspect 16. An implantable or insertable urological medical device comprising (a) a biostable device body comprising a first polymer and a first urologically beneficial agent and (b) a biodisintegrable coating comprising a second polymer and a second urologically beneficial agent disposed over the biostable device body, wherein the first and second polymers differ from one another, wherein the first and second urologically beneficial agents may be the same or different, and wherein upon implantation or insertion in a subject, the first urologically beneficial agent exhibits an extended release profile and the second urologically beneficial agent exhibits a rapid release profile.

Aspect 17. The urological medical device of aspect 16, wherein the coating is of a thickness sufficient to significantly increase the stiffness of the device relative to the stiffness of the device in the absence of the coating.

Aspect 18. The urological medical device of aspect 16, wherein the first and second urologically beneficial agents are different.

Aspect 19. The urological medical device of aspect 18, wherein the first urologically beneficial agent is an antimicrobial agent or a discomfort reducing agent, and wherein the second urologically beneficial agent is a discomfort reducing agent.

Aspect 20. The urological medical device of aspect 16, wherein the urological medical device is a ureteral stent comprising a lumen that extends along at least a portion of the length of the ureteral stent.

Aspect 21. The urological medical device of aspect 20, wherein the ureteral stent comprises a plurality of first openings that extend from the exterior of the stent to the lumen.

Aspect 22. An implantable or insertable urological medical device comprising a device body comprising (a) a biostable phase domain that comprises a first polymer and a first urologically beneficial agent and (b) a biodisintegrable phase domain that comprises a second polymer and a second urologically beneficial agent, wherein the first and second polymers differ from one another, wherein the first and second urologically beneficial agents may be the same or different, and wherein upon implantation or insertion in a subject, the first urologically beneficial agent exhibits an extended release profile and the second urologically beneficial agent exhibits a rapid release profile.

Aspect 23. The urological medical device of aspect 22, wherein the first and second urologically beneficial agents are different.

Aspect 24. The urological medical device of aspect 23, wherein the first urologically beneficial agent is an antimicrobial agent or a discomfort reducing agent, and wherein the second urologically beneficial agent is a discomfort reducing agent.

Aspect 25. The urological medical device of aspect 22, wherein the urological medical device is a ureteral stent comprising a lumen that extends along at least a portion of the length of the ureteral stent.

Aspect 26. The urological medical device of aspect 25, wherein the ureteral stent comprises a plurality of first openings that extend from the exterior of the stent to the lumen.

Aspect 27. An implantable or insertable urological medical device comprising a biostable elongate device body comprising a first polymer and a first urologically beneficial agent, the device body comprising a first lumen, which is at least partially lined with a porous layer, and a port between the first lumen and an exterior of the device, the port being adapted to allow the transfer of a solution or dispersion into the first lumen.

Aspect 28. The urological medical device of aspect 27, wherein the porous layer is formed by a process selected from a phase inversion process, a track-etch process or a laser ablation process.

Aspect 29. The urological medical device of aspect 27, wherein the first urologically beneficial agent is an antimicrobial agent or a discomfort reducing agent.

Aspect 30. The urological medical device of aspect 27, wherein the first polymer is EVA.

Aspect 31. The urological medical device of aspect 27, wherein the port further comprises a plug or septum.

Aspect 32. The urological medical device of aspect 27, comprising a plurality of openings extending between the exterior of the device and the first lumen.

Aspect 33. The urological medical device of aspect 27, wherein the urological medical device is a ureteral stent, wherein the first lumen extends along at least a portion of the length of the ureteral stent and wherein the ureteral stent comprises a second lumen extending along at least a portion of the length of the stent.

Aspect 34. The urological medical device of aspect 33, wherein the ureteral stent comprises a plurality of openings that extend from the exterior of the stent to the first lumen and a plurality of openings that extend from the exterior of the stent to the second lumen.

Aspect 35. A kit comprising the urological medical device of aspect 27 and a solution or dispersion of a second urologically beneficial agent.

Aspect 36. The kit of aspect 35, where the first urologically beneficial agent is an antimicrobial agent or a discomfort reducing agent and wherein the second urologically beneficial agent is a discomfort reducing agent.

Aspect 37. The kit of aspect 36, wherein upon introduction of the second urologically beneficial agent into the first lumen of the urological medical device though the port and upon the implantation or insertion of the urological medical device into a subject, the first urologically beneficial agent exhibits an extended release profile and the second urologically beneficial agent exhibits a rapid release profile.

### EXAMPLE 1

A biodissolvable polymer (e.g., a Klucel® polymer) and a urologically beneficial agent (e.g. ketorolac) are blended in a twin screw extruder to form pellets of a compounded biodisintegrable composition for further processing. Separately, a non-biodissolvable polymer (e.g., EVA), a urologically beneficial agent (e.g., triclosan or ketorolac), and an imaging agent (e.g., a radio-opacifying agent such as bismuth subcarbonate) are blended in a twin screw extruder to form pellets of a compounded biostable composition for further processing.

### EXAMPLE 2

Pellets of the biodisintegrable composition of Example 1 and pellets of the biostable composition of Example 1 are combined and extruded into tubes of various sizes (e.g., ranging from 5 to 8 Fr). The extruded tubes comprise a biodisintegrable polymeric phase domain comprising the Klucel® polymer and a biostable polymeric phase domain comprising the EVA polymer. The extruded material is cut to length and machined to provide drainage ports. If desired, the resulting tube may be machined to provide a tapered tip, and the tube may be annealed to create renal and bladder coils. A lubricious coating may also be provided as desired.

### EXAMPLE 3

Pellets of the biodisintegrable composition of Example 1 and pellets of the biostable composition of Example 1 are co-extruded into tubes of various sizes (e.g., ranging from 5 to 8 Fr), which have a biostable inner annular component (corresponding the biostable pellet material) and a biodisintegrable outer annular component (corresponding the biodisintegrable pellet material) (see, e.g., FIG. 7). The extruded material is cut to length and machined to provide drainage ports. If desired, the resulting tube may be machined to provide a tapered tip, and the tube may be annealed to create renal and bladder coils. A lubricious coating may also be provided as desired.

### EXAMPLE 4

Pellets of the biostable composition of Example 1 are extruded into dual lumen tubes (i.e., tubes having a drainage lumen and a drug delivery lumen) of various sizes (e.g., ranging from 5 to 8 Fr). The extruded material is cut to length and machined to provide drainage ports in fluid communication with the drainage lumen and drug delivery ports in fluid communication with the drug delivery lumen. If desired, the resulting tube may be machined to provide a tapered tip, and the tube may be annealed to create renal and bladder coils. A lubricious coating may also be provided as desired. Separately, pellets of the biodisintegrable composition of Example 1 are extruded into a solid rod or another form which is sized to be insertable into the drug delivery lumen of the tube.

Although various embodiments are specifically illustrated and described herein, it will be appreciated that modifications and variations of the present invention are covered by the above teachings and are within the purview of any appended claims without departing from the spirit and intended scope of the invention.

1. An implantable or insertable urological medical device comprising
(a) a biostable device body comprising a first polymer and a first urologically beneficial agent, said device body comprising a first lumen, and
(b) a biodisintegrable component comprising a second polymer and a second urologically beneficial agent disposed within said first lumen, wherein said first and second polymers differ from one another, wherein said first and second urologically beneficial agents may be the same or different, and wherein upon implantation or insertion in a subject, said first urologically beneficial agent exhibits an extended release profile and said second urologically beneficial agent exhibits a rapid release profile.

2. The urological medical device of claim 1, wherein said first and second urologically beneficial agents are different.

3. The urological medical device of claim 2, wherein said first urologically beneficial agent is an antimicrobial agent or a discomfort reducing agent, and wherein said second urologically beneficial agent is a discomfort reducing agent.

4. The urological medical device of claim 1, wherein said first polymer is EVA and said second polymer is a biodissolvable polymer.

5. The urological medical device of claim 1, wherein said first polymer is EVA and said second polymer is a biodissolvable cellulose.

6. The urological medical device of claim 5, wherein biodissolvable cellulose is selected from hydroxymethyl cellulose, hydroxyethyl cellulose, hydroxyproyl cellulose, and combinations thereof.

7. The urological medical device of claim 1, wherein said biodisintegrable component is in the form of an elongated rod.

8. The urological medical device of claim 1, comprising a plurality of biodisintegrable components.

9. The urological medical device of claim 8, wherein said biodisintegrable components are in the form of spheres.

10. The urological medical device of claim 1, further comprising an aperture extending from said first lumen to the exterior of said device, said aperture being sized to allow the introduction of said biodisintegrable component from the exterior of said device into said first lumen, and a plug that is sized to plug said aperture.

11. The urological medical device of claim 1, further comprising a plurality of first openings extending between the exterior of said device and said first lumen.

12. The urological medical device of claim 1, wherein said urological medical device is a ureteral stent and wherein said first lumen extends along at least a portion of the length of said ureteral stent.

13. The urological medical device of claim 12, wherein said ureteral stent comprises a second lumen extending along at least a portion of the length of said ureteral stent.

14. The urological medical device of claim 13, wherein said ureteral stent comprises a plurality of first openings that extend from the exterior of said stent to said first lumen and a plurality of second openings that extend from the exterior of said stent to said second lumen.

15. A kit comprising
(a) implantable or insertable urological medical device comprising a biostable device body that comprises a first polymer and a first urologically beneficial agent, said device body comprising a first lumen and an aperture between said first lumen and the exterior of the device, and
(b) a biodisintegrable component comprising a second polymer and a second urologically beneficial agent sized to fit through said aperture and into said first lumen, wherein said first and second polymers differ from one another, wherein said first and second urologically beneficial agents may be the same or different, and wherein upon implantation or insertion into a subject, said first urologically beneficial agent exhibits an extended release profile and said second urologically beneficial agent exhibits a rapid release profile.

16. An implantable or insertable urological medical device comprising
(a) a biostable device body comprising a first polymer and a first urologically beneficial agent, and
(b) a biodisintegrable coating comprising a second polymer and a second urologically beneficial agent disposed over said biostable device body, wherein said first and second polymers differ from one another, wherein said first and second urologically beneficial agents may be the same or different, and wherein upon implantation or insertion in a subject, said first urologically beneficial agent exhibits an extended release profile and said second urologically beneficial agent exhibits a rapid release profile.

17. The urological medical device of claim 16, wherein said coating is of a thickness sufficient to significantly increase the stiffness of said device relative to the stiffness of said device in the absence of the coating.

18. The urological medical device of claim 16, wherein said first and second urologically beneficial agents are different.

19. The urological medical device of claim 18, wherein said first urologically beneficial agent is an antimicrobial agent or a discomfort reducing agent, and wherein said second urologically beneficial agent is a discomfort reducing agent.

20. The urological medical device of claim 16, wherein said urological medical device is a ureteral stent comprising a lumen that extends along at least a portion of the length of said ureteral stent.

21. The urological medical device of claim 20, wherein said ureteral stent comprises a plurality of first openings that extend from the exterior of said stent to said lumen.

22. An implantable or insertable urological medical device comprising a device body comprising
(a) a biostable phase domain that comprises a first polymer and a first urologically beneficial agent, and
(b) a biodisintegrable phase domain that comprises a second polymer and a second urologically beneficial agent, wherein said first and second polymers differ from one another, wherein said first and second urologically beneficial agents may be the same or different, and wherein upon implantation or insertion in a subject, said first urologically beneficial agent exhibits an extended release profile and said second urologically beneficial agent exhibits a rapid release profile.

23. The urological medical device of claim 22, wherein said first and second urologically beneficial agents are different.

24. The urological medical device of claim 23, wherein said first urologically beneficial agent is an antimicrobial agent or a discomfort reducing agent, and wherein said second urologically beneficial agent is a discomfort reducing agent.

25. The urological medical device of claim 22, wherein said urological medical device is a ureteral stent comprising a lumen that extends along at least a portion of the length of said ureteral stent.

26. The urological medical device of claim 25, wherein said ureteral stent comprises a plurality of first openings that extend from the exterior of said stent to said lumen.

27. An implantable or insertable urological medical device comprising a biostable elongate device body comprising a first polymer and a first urologically beneficial agent, said device body comprising a first lumen, which is at least partially lined with a porous layer, and a port between said first lumen and an exterior of the device, said port being adapted to allow the transfer of a solution or dispersion into said first lumen.

28. The urological medical device of claim 27, wherein said porous layer is formed by a process selected from a phase inversion process, a track-etch process or a laser ablation process.

29. The urological medical device of claim 27, wherein said first urologically beneficial agent is an antimicrobial agent or a discomfort reducing agent.

30. The urological medical device of claim 27, wherein said first polymer is EVA.

31. The urological medical device of claim 27, wherein said port further comprises a plug or septum.

32. The urological medical device of claim 27, comprising a plurality of openings extending between the exterior of said device and said first lumen.

33. The urological medical device of claim 27, wherein said urological medical device is a ureteral stent, wherein said first lumen extends along at least a portion of the length of said ureteral stent and wherein said ureteral stent comprises a second lumen extending along at least a portion of the length of said stent.

34. The urological medical device of claim 33, wherein said ureteral stent comprises a plurality of openings that extend from the exterior of said stent to said first lumen and a plurality of openings that extend from the exterior of said stent to said second lumen.

35. A kit comprising said urological medical device of claim 27 and a solution or dispersion of a second urologically beneficial agent.

36. The kit of claim 35, where said first urologically beneficial agent is an antimicrobial agent or a discomfort reducing agent and wherein said second urologically beneficial agent is a discomfort reducing agent.

37. The kit of claim 36, wherein upon introduction of said second urologically beneficial agent into said first lumen of said urological medical device through said port and upon the implantation or insertion of said urological medical device into a subject, said first urologically beneficial agent exhibits an extended release profile and said second urologically beneficial agent exhibits a rapid release profile.
